(19) United States
(12) Patent Application Publication
(10) Pub. No.: US 2006/0073517 A1
(43) Pub. Date: Apr. 6, 2006

(54) Post-translational modifications of proteins as regulatory switches

(76) Inventors: C David Allis, New York, NY (US); Wolfgang Fishle, New York, NY (US); Yanming Wang, University Park, PA (US); Donald F Hunt, Charlottesville, VA (US)

Correspondence Address: UNIVERSITY OF VIRGINIA PATENT FOUNDATION, 250 WEST MAIN STREET, SUITE 300, CHARLOTTESVILLE, VA, 22902, US

(21) Appl. No.: 10/548,747

(22) PCT Filed: Mar. 9, 2004
(86) PCT No.: PCT/US04/07186

§ 371 Date: Sep. 8, 2005

Related U.S. Application Data
(60) Provisional application No. 60/453,358, filed on Mar. 10, 2003.

(51) Int. Cl. G01N 33/53; C07H 21/04; C12P 21/06; C12N 5/06; C07K 16/40

(52) U.S. Cl. 435/7.1; 435/69.1; 435/320.1; 435/326; 530/388.26; 536/23.53

(57) ABSTRACT

The present invention relates to the generation of antibodies that bind to specific modifications of the amino terminus of histone H3 and the carboxy terminus of the H2A and H2B peptides. More particularly, the present invention is directed to the generation of a set of antibodies that recognize various post-translational modifications of histone proteins that function as switches for regulating transcription and mitosis. Compositions comprising these antibodies are used as diagnostic and screening tools.

This application claims priority under 35 U.S.C. § 119(e) to US Provisional Patent application No. 60/453,358, filed on Mar. 10, 2003, the disclosure of which is incorporated herein by reference in its entirety.

### US GOVERNMENT RIGHTS

This invention was made with United States Government support under Grant Nos. GM53512, GM40922 and GM63959, awarded by the National Institutes of Health. The United States Government has certain rights in the invention.

### BACKGROUND

In eukaryotes, DNA is complexed with histone proteins to form nucleosomes, the repeating subunits of chromatin. This packaging of DNA imposes a severe restriction to proteins seeking access to DNA for DNA-templated processes such as transcription or replication. It is becoming increasingly clear that post-translational modifications of histone amino-termini play an important role in determining the chromatin structure of the eukaryotic cell genome as well as regulating the expression of cellular genes.

Posttranslational modifications of histone amino-termini have long been thought to play a central role in the control of chromatin structure and function. A large number of covalent modifications of histones have been documented, including acetylation, phosphorylation, methylation, ubiquitination, and ADP ribosylation, that take place on the amino terminus “tail” domains of histones. Such diversity in the types of modifications and the remarkable specificity for residues undergoing these modifications suggest a complex hierarchy of order and combinatorial function that remains unclear. Of the covalent modifications known to take place on histone amino-termini, acetylation is perhaps the best studied and appreciated. Recent studies have identified previously characterized coactivators and corepressors that acetylate or deacetylate, respectively, specific lysine residues in histones in response to their recruitment to target promoters in chromatin (See Berger (1999) Curr. Opin. Genet. Dev. 11, 336-341). These studies provide compelling evidence that chromatin remodeling plays a fundamental role in the regulation of transcription from nucleosomal templates.

The modest number of genes in the human genome has lead to the conclusion that there must be additional elements besides the nucleic acid sequence of the genome to account for the complexities inherent in human development and the sophisticated signaling systems that maintain homeostasis. Through the use of antibodies that specifically recognize post-translationally modified histones, applicants have been elucidating a “histone code.” In particular, evidence is emerging that histone proteins, and their associated covalent modifications, contribute to a mechanism that can alter chromatin structure, thereby leading to inherited differences in transcriptional “on-off ” states or to the stable propagation of chromosomes by defining a specialized higher-order structure. The degree of “openess” of chromatin structure and hence transcriptional activity is regulated by protein complexes that involve histone and DNA enzymatic modifications.

In addition to the previously described histone code, a new theme is emerging in the field that histone modifications most likely do not function in isolation. For example, phosphorylation of H3 at Ser10 appears to have a “split personality,” exhibiting clear links to mitotic chromatin as well as transcriptionally-active chromatin. These results seem counter-intuitive, but they have provided an impetus to explore if other neighboring modifications act together with Ser10 Phos H3 to distinguish these responses. Recent data, collected in yeast and in mammalian cells, document that Phos (Ser10) H3 can function in some circumstances with adjacent or nearby acetylation sites (Lys9 and/or Lys14). The exact order of addition or degree of non-randomness of these events remains controversial. Nevertheless, the Phos/Acetyl di-modification of H3 seems to be part of a highly conserved gene-inductive response.

The fact that post-translational modifications of adjacent amino acid residues may interact to produce an effect has lead to the notion that certain “histone modification cassettes” exist that regulate basic genomic functions such as transcription and replication. A histone modification cassette comprises a primary histone amino acid sequence that contains two or more sites that are naturally modified under certain circumstances, wherein the post-translational modifications interact to give a specific response.

In a further extension of this concept, applicants have discovered that covalent modification of an adjacent amino acid on a protein (such as histone “phos/methyl” or “methyl/phos” combinations) can function as “switches” to regulate gene expression and bioactivity of proteins. According to this model, modification of a particular site/mark results in the recruitment of a binding factor/module, whereas the modification of the adjacent amino acid leads to the loss of binding of the effector. The reversibility of the modification establishes a switch that effects regulation of the associated gene or bioactive protein.

The present invention is directed to an expanded histone code hypothesis from a largely linear viewpoint of single tail-restricted histone modifications to a more accurate accounting of the complex nature of chromatin (i.e. two copies of each histone per nucleosome, arrays/domains of nucleosomes as information units).

### SUMMARY OF THE INVENTION

The present invention is directed to antibodies that bind to specific modifications of the amino terminus of histone H3 and the carboxy terminus of histone H2A and H2B peptides. More particularly, the present invention is directed to the generation of a set of antibodies that recognize various post-translational modifications of a histone modification cassette or switch. Furthermore, these antibodies recognize epitopes on non-histone proteins that may be linked to human biology and disease. Compositions comprising these antibodies are used as diagnostic and screening tools.

### BRIEF DESCRIPTION OF THE DRAWINGS

FIG. 1 is a schematic representation of a “phos/methyl” or “methyl/phos” switch. Single marks are anticipated to recruit specific binding proteins X and Y (such as the chromodomains of HP1 on methylated H3-lys9). Known effector modules (bromo- and chromodomains) bind relatively weakly to their cognate marks (Kds in the low micromolar range). These low affinities allow for physiologically meaningful “on-off” binding reactions that impact the binding of effector proteins. Thus the switch (controling the binding of an effector module) can be turned on or off by a pair of kinases and phosphatases.

FIG. 2 is a graph demonstrating the 100× drop in binding affinity of HP1 for H3 after the serine 10 residue adjacent to the methylated lysine 9 residue gets phosphorylated.

FIG. 3 is a schematic representation of “off effectors” and “on effectors” and how they function in accordance with the “methyl-phos” switch.

FIG. 4 represents a Western blot of whole cell yeast extracts of various mutants cell lines, probed with the listed methyl-specific H3 antibodies. The results identify the specific histone methylase activity that is missing in the yeast deletion mutation extracts and thus identifies that deleted gene as the one that provides the relevant methylase activity in the wild type organism.

### DETAILED DESCRIPTION OF THE INVENTION

Definitions

In describing and claiming the invention, the following terminology will be used in accordance with the definitions set forth below.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

Naturally occurring amino acid residues include the following compounds (abbreviated as recommended by the IUPAC-IUB Biochemical Nomenclature Commission): Phenylalanine is Phe or F; Leucine is Leu or L; Isoleucine is Ile or I; Methionine is Met or M; Norleucine is NMe; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; Glycine is Gly or G.

As used herein, the term “conservative amino acid substitution” is defined herein as exchanges within one of the following five groups:
I. Small aliphatic, nonpolar or slightly polar residues:
   Ala, Ser, Thr, Pro, Gly,
II. Polar, negatively charged residues and their amides:
   Asp, Asn, Glu, Gln;
IV. Polar, positively charged residues:
   His, Arg, Lys;
IV. Large, aliphatic, nonpolar residues:
   Met Leu, Ile, Val, Cys
V. Large, aromatic residues:
   Phe, Tyr, Trp

As used herein the term “solid support” relates to a solvent insoluble substrate that is capable of forming linkages (preferably covalent bonds) with soluble molecules. The support can be either biological in nature, such as, without limitation, a cell or bacteriophage particle, or synthetic, such as, without limitation, an acrylamide derivative, glass, plastic, agarose, cellulose, nylon, silica, or magnetized particles. The surface of such supports may be solid or porous and of any convenient shape.

The term “linked” or like terms refers to the connection between two groups. The linkage may comprise a covalent, ionic, or hydrogen bond or other interaction that binds two compounds or substances to one another.

As used herein, the term “treating” includes alleviating the symptoms associated with a specific disorder or condition and/or preventing or eliminating said symptoms. For example, treating cancer includes preventing or slowing the growth and/or division of cancer cells as well as killing cancer cells.

As used herein, the term “histone modification cassette” is intended to include any grouping of two or more histone modifications within a contiguous amino acid sequence of a histone tail that in combination are associated with a specific biological response. Examples of specific biological responses include but are not limited to transcriptional activation and the initiation of mitosis or meiosis.

As used herein, the term “antibody” refers to a polyclonal or monoclonal antibody or a binding fragment thereof (that retains the specific binding of the whole antibody) such as Fab, F(ab′)2 and Fv fragments.

As used herein, the term “biologically active fragments” of the antibodies described herein encompasses natural or synthetic portions of the respective full-length antibody that retain the capability of specific binding to the target epitope.

As used herein, the term “parenteral” includes administration subcutaneously, intravenously or intramuscularly.

The term “modified amino acid” as used herein includes a natural amino acid residue comprising one or more modifying groups covalently bound to the amino acid. For example, each modified lysine residue has the capacity to be mono-, di-, or tri-methylated, and the general reference to a methylated lysine is intended to encompass all three of these possibilities.

As used herein the designations “T^{(P)}”, “S^{(P)}”, “K^{(M)}” and “K^{(U)}” and like terms, represent modified amino acids, and more particularly, a phosphorylated threonine, a phosphorylated serine, a methylated lysine and a ubiquinated lysine, respectively. Furthermore, it is understood that these designations represent an amino acid that contains at least one of the designated modifying groups.

The Invention

One embodiment of the present invention is directed to compositions and methods for identifying transcriptionally active and inactive regions of chromatin and the use of such information for diagnostic and therapeutic purposes. Compositions encompassed by the present invention comprise antibodies that are specific for certain post-translational modifications of histone proteins wherein the modifications have been associated with a biological state. More particularly, one embodiment the present invention is directed to antibodies that recognize various post-translational modifications of histone proteins wherein the modifications function to act as switches for regulating transcription and/or mitotic activity. Compositions comprising these antibodies are used as diagnostic and screening tools.

In accordance with one embodiment antibodies are generated that specifically bind to modified amino acids present in the amino terminus of histone H3 and the carboxy terminus of histone H2A and H2B peptides. More particularly, antibodies are generated using a composition comprising a polypeptide wherein the polypeptide comprises an amino acid sequence selected from the group consisting of:

| | |
|---|---|
| ART^{(P)}KQ, | (SEQ ID NO: 1) |
| ART^{(P)}K^{(M)}Q, | (SEQ ID NO: 2) |
| ARKS^{(P)}T, | (SEQ ID NO: 3) |
| ARK^{(M)}S^{(P)}T, | (SEQ ID NO: 4) |
| LAT^{(P)}KA, | (SEQ ID NO: 5) |
| LAT^{(P)}K^{(M)}A, | (SEQ ID NO: 6) |
| LATK^{(M)}A, | (SEQ ID NO: 7) |
| ARKS^{(P)}A, | (SEQ ID NO: 8) |
| ARK^{(M)}S^{(P)}A, | (SEQ ID NO: 9) |
| ARK^{(M)}SA, | (SEQ ID NO: 10) |
| PKKT^{(P)}E, | (SEQ ID NO: 11) |
| PKK^{(U)}T^{(P)}E, | (SEQ ID NO: 12) |
| PKK^{(U)}TE, | (SEQ ID NO: 13) |
| AVT^{(P)}KY, | (SEQ ID NO: 14) |
| AVT^{(P)}K^{(U)}Y, | (SEQ ID NO: 15) |
| AVTK^{(U)}Y, | (SEQ ID NO: 16) |

wherein the symbols “T^{(P)}”, “S^{(P)}”, “K^{(M)}” and “K^{(U)}” represent a phosphorylated threonine, a phosphorylated serine, a methylated lysine and a ubiquinated lysine, respectively. In another embodiment an antigenic composition is provided that comprises a polypeptide wherein the polypeptide comprises an amino acid sequence selected from the group consisting of:

| | |
|---|---|
| DFK^{(M)}TD, | (SEQ ID NO: 20) |
| DFK^{(M)}T^{(P)}D, | (SEQ ID NO: 21) |
| DFKT^{(P)}D, | (SEQ ID NO: 22) |
| KRK^{(M)}TV, | (SEQ ID NO: 23) |
| KRK^{(M)}T^{(P)}V, | (SEQ ID NO: 24) |
| and | |
| KRKT^{(P)}V. | (SEQ ID NO: 25) |

SEQ ID NOs: 1 and 2 represent modifications of amino acids 2 and 3 of H3; SEQ ID NOs: 3 and 4 represent modifications of amino acids 9 and 10 of H3; SEQ ID NOs: 5-7 represent modifications of amino acids 22 and 23 of H3; SEQ ID NOs: 8-10 represent modifications of amino acids 27 and 28 of H3; SEQ ID NOs: 11-13represent modifications of amino acids 119 and 120 of H2A; and SEQ ID NOs: 1416 represent modifications of amino acids 119 and 120 of H2B. SEQ ID NOs: 20-22 represent modifications of amino acids 79 and 80 of H3. SEQ ID NOs: 23-25 represent modifications of amino acids 79 and 80 of H4.

In one embodiment the polypeptide of the present compositions is a purified antigenic fragment of a histone protein, or a corresponding synthetic equivalent thereof. More particularly, the antigenic polypeptide comprises a 9 to 20 amino acid sequence, and in one embodiment a 9 amino acid sequence, wherein the amino acid sequence comprises the sequence of SEQ ID NOs: 1-16, or an amino acid sequence that differs from an amino acid sequence of SEQ ID NOs: 1- 16 by a single conservative amino acid substitution. In an alternative embodiment, the purified antigen comprises an amino acid sequence selected from the group of SEQ ID NOs: 1-16 and 20-25 linked to a suitable carrier, such as bovine serum albumin or Keyhole limpet hemocyanin.

The present invention also encompasses antibodies generated against the modified peptides of SEQ ID NOs. 1-16 and 20-25. One method used to generate these antibodies involves administration of the respective antigens to a laboratory animal, typically a rabbit, to trigger production of antibodies specific for the antigen. Accordingly, the present invention also encompasses antigenic compositions comprising a polypeptide, wherein the polypeptide comprises an amino acid sequence selected from the group consisting of SEQ ID NO: 1-16 and 20-25 and a pharmaceutically acceptable carrier. The composition may further comprise diluents, excipients, solubilizing agents, stabilizers and adjuvants. Carriers and diluents suitable for use with the present invention include sterile liquids such as water and oils. Illustrative oils are those of petroleum, animal, vegetable, or synthetic origin, for example, peanut oil, soybean oil, or mineral oil. In general, water, saline, aqueous dextrose, and related sugar solution, and glycols such as, propylene glycol or polyethylene glycol, are preferred liquid carriers, particularly for injectable solutions. Suitable adjuvants include alum or complete Freund's adjuvant (such as Montanide ISA-51).

The dose and regiment of antigen administration necessary to trigger antibody production as well as the methods for purification of the antibody are well known to those skilled in the art. Typically, such antibodies can be raised by administering the antigen of interest subcutaneously to New Zealand white rabbits which have first been bled to obtain pre-immune serum. The antigens can be injected at a total volume of 100 ul per site at six different sites. Each injected material will contain synthetic surfactant adjuvant pluronic polyols, or pulverized acrylamide gel containing the protein or polypeptide after SDS-polyacrylamide gel electrophoresis.

The rabbits are then bled two weeks after the first injection and periodically boosted with the same antigen three times every six weeks. A sample of serum is then collected 10 days after each boost Polyclonal antibodies are then recovered from the serum by affinity chromatography using the corresponding antigen to capture the antibody. Ultimately, the rabbits are euthenized, for example with pentobarbital 150 mg/Kg IV. This and other procedures for raising polyclonal antibodies are disclosed in E. Harlow, et al., editors, Antibodies: A Laboratory Manual (1988), which is hereby incorporated by reference. The specificity of antibodies may be determined by enzyme-linked immunosorbent assay or inumunoblotting, or similar methods known to those skilled in the art.

One aspect of the present invention is directed to antibodies that specifically bind to a polypeptide comprising an amino acid sequence selected from the group consisting of:

| | |
|---|---|
| ART^{(P)}KQ, | (SEQ ID NO: 1) |
| ART^{(P)}K^{(M)}Q, | (SEQ ID NO: 2) |
| ARKS^{(P)}T, | (SEQ ID NO: 3) |
| ARK^{(M)}S^{(P)}T, | (SEQ ID NO: 4) |
| LAT^{(P)}KA, | (SEQ ID NO: 5) |
| LAT^{(P)}K^{(M)}A, | (SEQ ID NO: 6) |
| LATK^{(M)}A, | (SEQ ID NO: 7) |
| ARKS^{(P)}A, | (SEQ ID NO: 8) |
| ARK^{(M)}S^{(P)}A, | (SEQ ID NO: 9) |
| ARK^{(M)}SA, | (SEQ ID NO: 10) |
| PKKT^{(P)}E, | (SEQ ID NO: 11) |
| PKK^{(U)}T^{(P)}E, | (SEQ ID NO: 12) |
| PKK^{(U)}TE, | (SEQ ID NO: 13) |
| AVT^{(P)}KY, | (SEQ ID NO: 14) |
| AVT^{(P)}K^{(U)}Y, | (SEQ ID NO: 15) |
| and | |
| AVTK^{(U)}Y. | (SEQ ID NO: 16) |

In one embodiment the antibodies of the present invention specifically bind to a polypeptide comprising an amino acid sequence selected from the group consisting of

| | |
|---|---|
| DFK^{(M)}TD, | (SEQ ID NO: 20) |
| DFK^{(M)}T^{(P)}D, | (SEQ ID NO: 21) |
| DFKT^{(P)}D, | (SEQ ID NO: 22) |
| KRK^{(M)}TV, | (SEQ ID NO: 23) |
| KRK^{(M)}T^{(P)}V, | (SEQ ID NO: 24) |
| and | |
| KRKT^{(P)}V. | (SEQ ID NO: 25) |

In one embodiment the antibodies of the present invention specifically bind to a polypeptide comprising an amino acid sequence selected from the group consisting of

| | |
|---|---|
| ART^{(P)}KQ, | (SEQ ID NO: 1) |
| ART^{(P)}K^{(M)}Q, | (SEQ ID NO: 2) |
| ARKS^{(P)}T, | (SEQ ID NO: 3) |
| ARK^{(M)}S^{(P)}T, | (SEQ ID NO: 4) |
| LAT^{(P)}KA, | (SEQ ID NO: 5) |
| LAT^{(P)}K^{(M)}A, | (SEQ ID NO: 6) |
| LATK^{(M)}A, | (SEQ ID NO: 7) |
| ARKS^{(P)}A, | (SEQ ID NO: 8) |
| ARK^{(M)}S^{(P)}A, | (SEQ ID NO: 9) |
| and | |
| ARK^{(M)}SA. | (SEQ ID NO: 10) |

In another embodiment the antibodies of the present invention specifically bind to a polypeptide comprising an amino acid sequence selected from the group consisting of

| | |
|---|---|
| PKKT^{(P)}E, | (SEQ ID NO: 11) |
| PKK^{(U)}T^{(P)}E, | (SEQ ID NO: 12) |
| PKK^{(U)}TE, | (SEQ ID NO: 13) |
| AVT^{(P)}KY, | (SEQ ID NO: 14) |
| AVT^{(P)}K^{(U)}Y, | (SEQ ID NO: 15) |
| and | |
| AVTK^{(U)}Y. | (SEQ ID NO: 16) |

As used herein, an antibody that binds specifically to a target antigen is an antibody that will produce a detectable signal in the presence of the target antigen but will not cross react with other non-target antigens (i.e. produces no detectable signal) under the identical conditions used to detect the target antigen. For example a monoclonal antibody generated against ART^{(P)}KQ (SEQ ID NO: 1) will not bind to the sequence ARTKQ (SEQ ID NO: 17) or any of the other peptides of SEQ ID NO: 2-16, when optimal conditions are used.

In one embodiment the antibodies generated against the target modified peptides are monoclonal antibodies. Monoclonal antibody production may be effected using techniques well-known to those skilled in the art. Basically, the process involves first obtaining immune cells (lymphocytes) from the spleen of a mammal (e.g., mouse) which has been previously immunized with the antigen of interest either in vivo or in vitro. The antibody-secreting lymphocytes are then fused with myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. One embodiment of the invention is directed to a hybridoma cell line which produces monoclonal antibodies which bind one of the target antigens of SEQ ID NO: 1-16 or 20-25. A description of the theoretical basis and practical methodology of fusing such cells is set forth in Kohler and Milstein, Nature, 256:495 (1975), which is hereby incorporated by reference.

In addition to whole antibodies, fragments of antibodies can retain binding specificity for a particular antigen. Antibody fragments can be generated by several methods, including, but not limited to proteolysis or synthesis using recombinant DNA technology. An example of such an embodiment is selective proteolysis of an antibody by papain to generate Fab fragments, or by pepsin to generate a F(ab′)2 fragment. These antibody fragments can be made by conventional procedures, as described in J. Goding, Monoclonal Antibodies: Principles and Practice, pp. 98-118 (N.Y. Academic Press 1983), which is hereby incorporated by reference. Other fragments of the present antibodies that retain the specific binding of the whole antibody can be generated by other means known to those skilled in the art.

The antibodies or antibody fragments of the present invention can be combined with a carrier or diluent to form a composition. These compositions can be used in standard Molecular Biology techniques such as Western blot analyses, immunofluorescence, and immunoprecipitation. In accordance with one embodiment the antibodies of the present invention are labeled for use in diagnostics or therapeutics. It is not intended that the present invention be limited to any particular detection system or label. The antibody may be labeled with a radioisotope, such as ³⁵S, ¹³¹I, ¹¹¹In, ¹²³I, ⁹⁹mTc, ³²P, ¹²⁵I, ³H, ¹⁴C, and ¹⁸⁸Rh, or a non-isotopic labeling reagent including fluorescent labels, such as fluorescein and rhodamine, or other non-isotopic labeling reagents such as biotin or digoxigenin. Antibodies containing biotin may be detected using “detection reagents” such as avidin conjugated to any desirable label such as a fluorochrome.

Additional labels suitable for use in accordance with the present invention include nuclear magnetic resonance active labels, electron dense or radiopaque materials, positron emitting isotopes detectable by a positron emission tomography (“PET”) scanner, chemilluminescers such as luciferin, and enzymatic markers such as peroxidase or phosphatase. In one embodiment the histone specific antibodies of the present invention are detected through the use of a secondary antibody, wherein the secondary antibody is labeled and is specific for the primary antibody. Alternatively, the antibodies of the present invention may be directly labeled with a radioisotope or fluorochrome such as FITC or rhodamine; in such cases secondary detection reagents may not be required for the detection of the labeled probe. In accordance with one embodiment the antibody is labeled with a fluorophore or chromophore using standard moieties known in the art.

The modified peptides of SEQ ID NOs: 1-16 and 20-25 have been identified as localized binary switches and/or modification cassettes that are believed to govern the biological readout of distinct modification patterns. More particularly, these modifications are believed to control effector-histone interactions and mediate critical biological functions. Monitoring the status of the “switch” provides information regarding the staus of the underlying DNA (active vs inactive).

Modification Cassettes

The concept of single and multiple covalent post-translational modifications embedded in histone proteins is now widely appreciated. Equally well documented is the existence of chromatin effector modules that “read” particular marks in histone tails (e.g. bromodomains or chromodomains that engage acetyl- or methyl-lysines, respectively, in the contexts of histone tail sequence). Expanding on these formal concepts is the concept of “modification cassettes” referring to a linear string of different clustered “marks” that may dictate distinct biological read-outs. Clusters of adjacent or closely-spaced modifiable residues are found in almost all core histones in multiple places along histone tails. For example, note the extreme amino terminus of most H4s-residues 1-5 [SGRGK], or more internally, residues 16-20 [KRHRK]. These are potential modificaton cassettes as Ser1, Arg3 and Lys5 of the H4 tail are well-known sites of phophorylation, methylation and acetylation, respectively. As well, Lys16 and Lys20 in the H4 tail are well-known sites of acetylation and methylation, respectively, and an older literature suggested that His18 may be an acid-labile phosphorylation site. Thus, both of these short stretches of the H4 tail contain an usually high density of distinct modification marks (3/5 residues are potentially covalently modified).

Interestingly, similar putative modification cassettes (containing the identical peptide sequences) can be found in other histone and non-histone proteins arguing for conserved functions that remain unclear. For example, the extreme N-terminus of H4 in most species [SGRGK] is also found at the very N-terminus of H2A of most, but not all, H2A family members. The generality of modification cassettes extending outside of histone proteins is underscored by several sting examples. The extreme N-terminus of Drosophila H4 [TGRGK] is also found in extreme N-tail Drosophila Polycomb, and here, remarkable similarity to the H4 tail extends well past the first five amino acids. This observation suggests the intriguing possibility that the “H4 tail” found in Polycomb contains a modification cassette that is used as part of a regulatory function that remains to be determined.

Local Switches

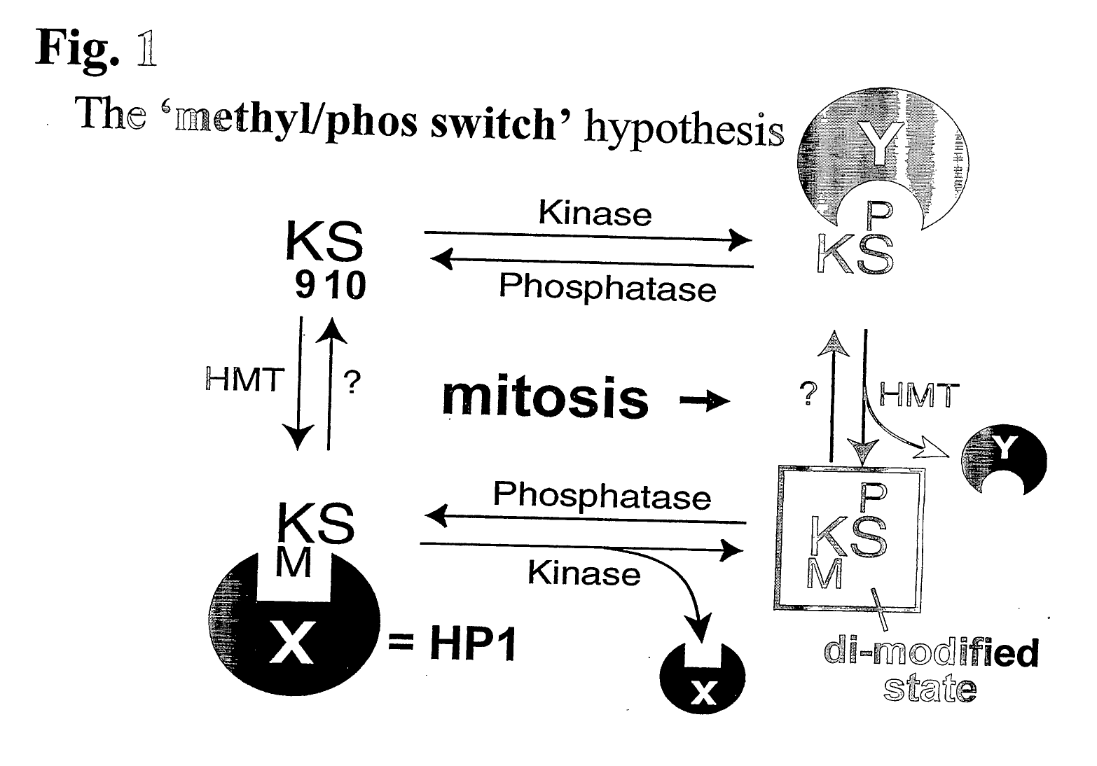

The concept of “local switches” is an intriguing extension of “modification cassettes.” According to this model, “local switches” involve the covalent modification of two adjacent amino acids, wherein modification of a first site recruits a binding factor/module, whereas modification of the second site leads to loss of the effector. In this model the modifications are fully reversible such that the binding of the effector is regulated by the post-translational modification of the histone protein. A readout of distinct combinations of marks in “modification cassettes” could be achieved by local switching mechanisms such as the “phos/methyl” or “methylphos” switch proposed in FIG. 1. Although the model shown in FIG. 1 is a “methyl/phos” switch, additional switches are envisioned such as “acetyl/phos” and “ubiquitin/phos” switches. A remarkably large number of sites of methylation and/or acetylation in H3 (K4, K9, K27 and K79, but not K14, K18 and K36) are next to known or potential sites of phosphorylation (TK/KT or SK/KS motifs). As well, the well-known sites of ubiquitin addition (K119 and K120 in human H2A and H2B, respectively) are also immediately adjactent to threonine residues suggesting that a similar switching mechanism my influence ubiquitin metabolism. Similar arrangements of potential modification “switches” exist in the histone fold domains of H2A, H2B and H4 and are mostly located at the edges of helical stretches or in the connecting loops. This concept can also be extended to potential “phos/acetyl”, “acetyl/phos”, “phos/ubiquitin” or “ubiquitin/phos” switches, or conceivably, any other patches with two or more covalent marks in close proximity.

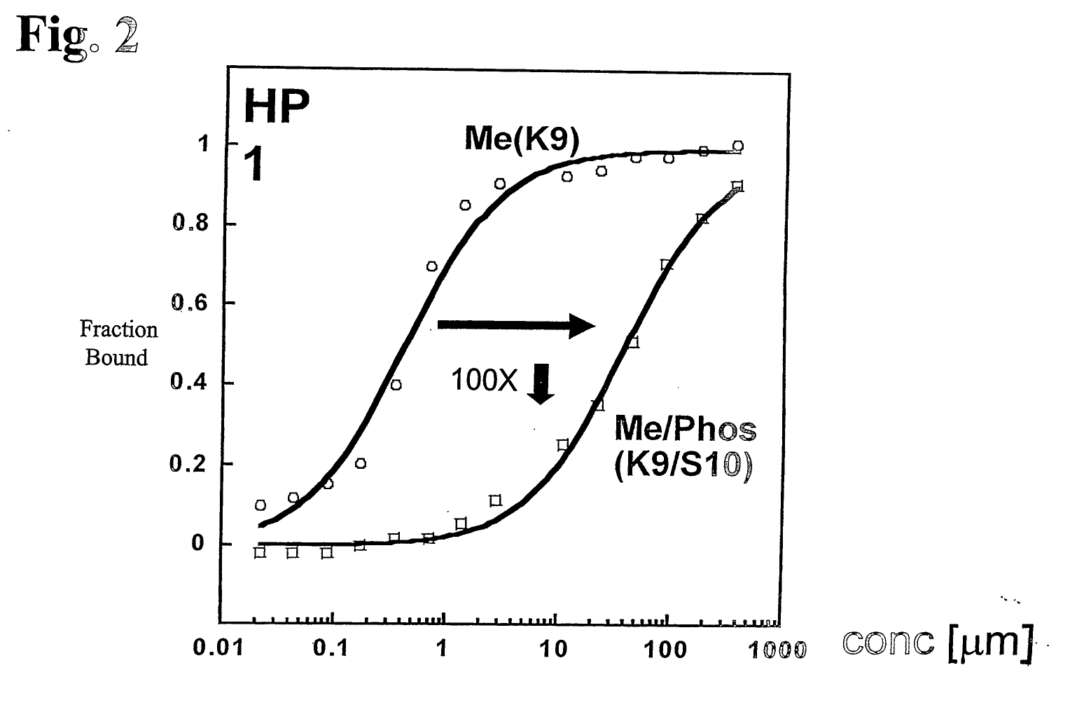

According to the structural analysis of the chromodomain of HP1 bound to the H3 tail methylated on K9, it can be deduced that phosphorylation of S10 will severely diminish the binding affinity of this module (See FIG. 2). The weak binding of binding factors to their cognate marks (Kds in the low micromolar range, i.e. approximately 10⁻⁴ to about 10⁻⁶ for both chromodomain/methyl-lysine and bromodomain/acetyl-lysine interactions) could allow for “on-off” binding, which could be further regulated by the nearby or adjacent modification of another site in the “off”-state. Such a kinetically controlled binding mechanism of effectors modules is sustained by the recent demonstration that binding proteins of heterochromatic methyl marks have a fast exchange rate with their environment and are not statically bound to their target sites.

In particular, “binary switches” could add an important dynamic nature to the readout of marks that themselves have very low turnover rates. For example, it is currently not understood if and how methyl marks in histones are reversed, and histone-demethylating enzymes are unknown. Some histone methyl-lysine marks appear to be stably maintained over several cell generations; yet, it is conceivable and likely that proteins bound to methyl marks are removed at some point in the cell cycle or during development of a complex organism. However in light of the stability of methyl marks, “methyl/phos switching” could be a widespread mechanism regulating the binding and release of effector modules to more stable methyl marks. In particular, the methyl mark may represent the more stable mark with an adjacent phosphorylation mark determining whether or not an effector protein binds. Furthermore, the binding of yet to be discovered phospho-binding effector modules (“Y” in FIG. 1) could also be regulated by nearby or adjacent “off” switches.

A site where a binary “methyl/phos switch” is anticipated to be operational is the Lys 9/Ser 10 region of H3. Methylation of Lys 9 by SET-type histone methyltransferases (HMT) like Su(Var)3-9 and G9a has been well documented and is associated with the establishment and maintenance of heterochromatic domains in many organisms. The chromodomain of HPI binds specifically to this mark and local HP1 recruitment is sufficient for mediating heterochromatin formation and accompanies gene silencing. Genome-wide mitotic phosphorylation of H3 Ser 10 is catalysed by aurora B-type kinases, and several other enzymes mediate a more localized and targeted employment of phospho-Ser 10 in response to immediate-early gene signalling. Whereas initial reports using enzymatic in vitro assays pointed to a mutually exclusive existence of the methyl-Lys 9 and phospho-Ser 10 marks, analysis of the in vivo modification pattern of H3 isolated from HeLa cells points to the coexistence of both marks on the same histone tail—especially during mitosis.

Structural examination of the chromodomain of HP1 bound to the H3 tail methylated on Lys 9, allows the prediction that additional phosphorylation of Ser 10 could severely diminish the binding affinity of BP to its cognate mark (that is, release of “X” in FIG. 1). Indeed, in in vitro assays chromodomains show almost complete loss of binding to dual modified peptides containing the methyl-Lys 9 and phospho-Ser 10 marks. If the general concept depicted in FIG. 1 is correct, mitosis (or meiosis) or pathways of gene activation may drive the phosphorylation of the proposed “methyl/phos switch,” allowing for the release and potential clearing of “negative” chromatin effectors like HP1 that repress transcription. This sequence of events could consecutively permit the docking of positive effectors that drive transcription (for example, HAT-containing complexes).

In support of the “methyl/phos switch” providing a mechanism for the release and potential clearing of “negative” chromatin effectors like HP1, HP1 is partially liberated from interphase heterochromatin domains as cells enter mitosis, a cell cycle stage marked by a rapid, transient burst of H3 Ser 10 phosphorylation. Furthermore, HP1 displays an increased mobility in T cells after receptor-driven kinase signalling is activated, an observation that is paralleled by a decrease of the immobile fraction of the protein.

The docking of effectors to post-translationally modified chromatin is reminiscent of the modular interactions in other signaling pathways (see for example the recruitment of SH2 domains to phosphor tyrosines). Bromodomains present in several HATs and chromatin remodeling proteins, as well as in the general transcription factor TAF250 bind acetylated lysines. Sequential recruitment and anchoring of bromodomain-containing factors and complexes to the promoter region is indeed crucial for the activation of some genes. Proteins containing certain chromodomains, on the other hand, have been predicted to have affinty for methylated lysines. In fact, as described above heterochromatin protein 1 (HP 1) can bind to methylated H3-K9, and more recent work suggests that the silencing protein Polycomb (Pc) can bind methylated H3-K9 and/or methylated H3-K27. It will be interesting to determine if other chromodomain-containing proteins bind yet other sites of lysine methylation in histones, or potentially, in non-histone proteins. Considering the enormous variability of histone modifications, it is likely that a number of other recognition modules still await discovery. For example, it is not known what docking modules, if any, bind to phosphoserines/threonines, methyl-arginines, etc. in the context of histones. Conversely, certain histone modifications or modification patterns appear to prevent the binding of chromatin-associated mediators or effector modules. Such “exclusion/repulsion” has been shown in the case of methylation of H3 on K4, which results in reduced binding of a chromatin repressor complex to the H3 tail. In addition, methylation of H3-K4 may inhibit the recruitment of repressive factors such as Pc and HP1 to H3-K9-methyl. These findings are consistent with the notion that methylation of H3 on K4 is generally believed to be an activating mark in higher organisms. Similarly, it has been suggested that methylation of H3-K79 in budding yeast prevents the spreading of silenced heterochromatic regions by preventing the binding of silencing proteins/complexes such as Sir2 to nucleosomes.

Additional support for a Lys 9/Ser 10 “methyl/phos switch” is derived from the finding that type 1 protein phosphatase (PP1) acts as a mitotic phosphatase targeting phospho-Ser 10 as cells exit mitosis. PP1 has independently been identified in genetic screens in Drosophila as Su(Var)3-6. Genes of the Su(Var) family facilitate heterochromatic gene silencing as assayed by suppression of position effect variegation by mechanisms that have remained elusive for a long time. It is anticipated that PP 1 removes mitotic phosphates at Ser 10 in H3, thus allowing increased binding of HP1 [genetically identified as Su(Var)2-5] to H3 methyl-Lys 9 marks, which are themselves added by a H3 Lys 9 methyltransferase (Su(Var)3-9).

There exist additional methyl-mark-specific modules whose binding is potentially sensitive to neighbouring phospho-marks. The best-studied methyl marks in the H3 tail (Lys 4, Lys 9 and Lys 27), for example, are all adjacent to novel (Thr 3) or previously described (Ser 10 and Ser 28) phospho-acceptors. Whereas the chromodomain of Polycomb preferentially binds the Lys 27 methyl mark, a module that “reads” the H3 Lys 4 methyl mark (a mark associated with an “on” or “competent” transcriptional state) has yet to be identified. If such a module exists, it follows that its binding to the H3 tail could be regulated by phosphorylation of Thr 3. Lys 23 in the H3 tail is another methylation site, and phosphorylation of Thr 22 could regulate the biology of this mark. Accordingly, up to four “methyl/phos switches” might be operating on the H3 tail alone and in one embodiment of the present invention antigenic peptides and corresponding antibodies are generated to the four “methyl/phos switches” H3 tail sites. More particularly, antibodies are generated using a composition comprising an amino acid sequence selected from the group consisting of:

| | |
|---|---|
| ART^{(P)}KQ, | (SEQ ID NO: 1) |
| ART^{(P)}K^{(M)}Q, | (SEQ ID NO: 2) |
| ARTK^{(M)}Q, | (SEQ ID NO: 18) |
| ARKS^{(P)}T, | (SEQ ID NO: 3) |
| ARK^{(M)}S^{(P)}T, | (SEQ ID NO: 4) |
| ARK^{(M)}ST, | (SEQ ID NO: 19) |
| LAT^{(P)}KA, | (SEQ ID NO: 5) |
| LAT^{(P)}K^{(M)}A, | (SEQ ID NO: 6) |
| LATK^{(M)}A, | (SEQ ID NO: 7) |
| ARKS^{(P)}A, | (SEQ ID NO: 8) |
| ARK^{(M)}S^{(P)}A, | (SEQ ID NO: 9) |
| ARK^{(M)}SA, | (SEQ ID NO: 10) |

In support of the suggested switch model, the Lys79 of H3 stands as the first known methylation site outside of a histone tail. It also lies adjacent to a potential phosphorylation site, Thr 80, and recent genetic screens have implied Lys 79 Thr 80 in a genomic “silencing cluster.” This cluster involves a corresponding region of H4, Lys 79 and Thr 80, suggesting that “methyl/phos switches” might regulate the critical interface between H3 and H4 (it has not been confirmed that Lys 79 of H4 is methylated and/or Thr 80 of H4 is phosphorylated). The idea that a “methyl/phos switch” may operate on a critical interface of the H3:H4 dimer is attractive given the importance of this boundary for nucleosome structure and gene regulation. The “silencing cluster” in H3 and H4 provides an excellent example of the second dimension of the histone code, a code that minimally operates at a nucleosome level. In accordance with one embodiment antigenic peptide and corresponding antibodies are generated to the modified Lys79 and Thr80 sites. More particularly, antibodies are generated using a composition comprising an amino acid sequence selected from the group consisting of:

| | |
|---|---|
| DFK^{(M)}TD, | (SEQ ID NO: 20) |
| DFK^{(M)}T^{(P)}D, | (SEQ ID NO: 21) |
| DFKT^{(P)}D, | (SEQ ID NO: 22) |
| KRK^{(M)}TV, | (SEQ ID NO: 23) |
| KRK^{(M)}T^{(P)}V, | (SEQ ID NO: 24) |
| and | |
| KRKT^{(P)}V. | (SEQ ID NO: 25) |

All potential “switches” identified to date are located either in the exposed histone tails, at the edges of helical stretches, or in the connecting loops of the histone fold domains. These sites could therefore all be accessible to post- translational modification and function as potential “switches.” The well-known ubiquitination sites (Lys 119 and Lys 120 in human H2A and H2B, respectively) are also adjacent to threonine residues, suggesting that switching mechanisms might influence ubiquitin attachment or removal, and/or the readout of ubiquityl-marks.

The discovery of monoubiquitination of H2B in yeast enabled genetic studies of histone ubiquitination. Surprisingly, mutagenesis of either the ubiquitin acceptor site, H2B-K123 (equals human H2B-K120), or disruption of the ubiquitin- conjugating enzyme Rad6/Ubc2 in this organism results in a striking loss of methylation at H3-K4 and H3-K79. Altogether, these results indicate that ubiquitination of H2B is a prerequisite for methylation of H3 on K4 and K79. On the other hand, abolishment of H3-K4 or H3-K79 methylation has no effect on H2B ubiquitination, suggesting that the cross-talk is unidirectional. This control of a modification pattern in “trans” is site-specific since another site of methylation of H3 in yeast, K36, is not affected (note: methylation of H3-K79 has not been detected in budding yeast.

Interestingly, inter-histone cross-talk may not be restricted to a single nucleosome. In yeast, about 5% of H2B is estimated to be ubiquitinated, about 35% of the total H3 pool is thought to be methylated on K4, and 90% of all H3 is methylated on K79. Since ubiquitination of H2B appears to be far sub-stochiometric to the methylation of H3, the newly discovered control mechanism might serve as a paradigm for “master control switches” directing the modification pattern of a whole nucleosomal region.

In addition to a methyl-lysine binary switch, similar switches could also be operational for more labile marks (that is, “acetyl/phos” or “ubiquitin/phos” switches). In keeping with the concept of local switches, mitotic phosphorylation of Ser10 and/or Ser28 in the H3 tail may be used to dislodge Lys9 (or Lys27) methyl-binding chromodomains like HP1 or Polycomb from mitotic chromosomes. Detailed mass spectrometric analyses of patterns of modifications in H3 peptides spanning Lys9/Ser10 or Lys27/Ser28 would be useful as cells enter and exit mitosis. Mitotic phosphorylation at Ser10 and/or Ser28 is well documented by immunocytological analyses. However, the status of adjacent methylation at Lys9 and/or Lys27 is unclear because of potential issues involving epitope disruption or occlusion. Equally unclear, is whether “mitotic” effector molecules exist that bind to phosphorylated Ser10 and/or Ser28. If so, these mitotic effectors may be sensitive to “off” switches that might involve nearby methylation marks (i.e. Lys9 and/or Lys27).

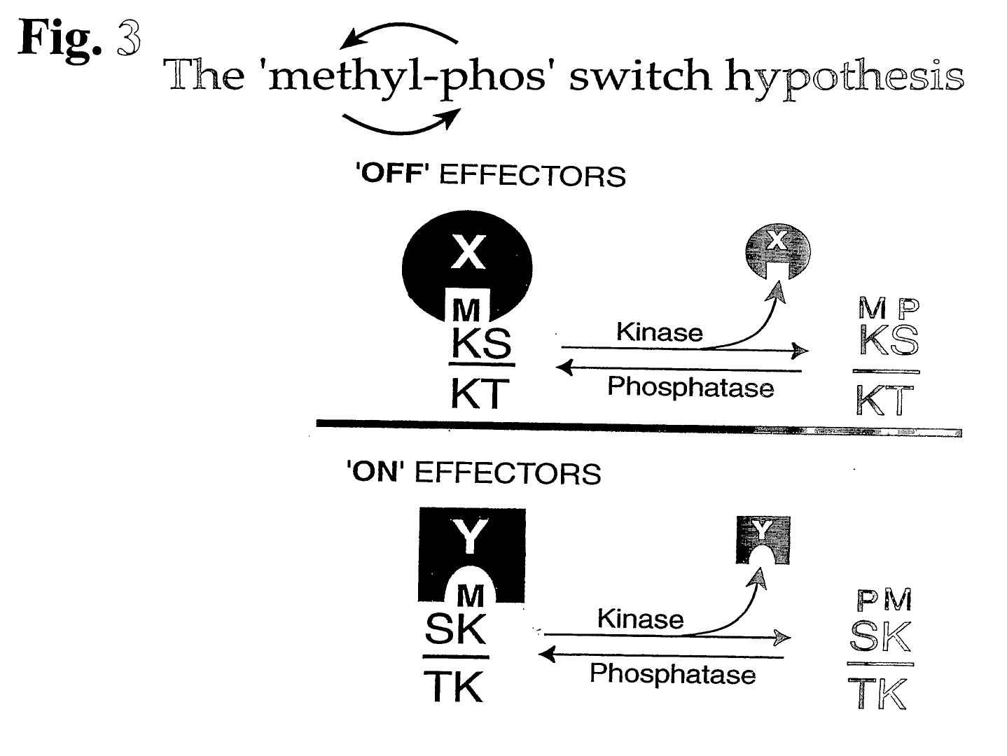

Putative lysine “switch” sites that have by now been implicated in binding repressive modules (Lys 9/Ser 10 and Lys 27/Ser 28 in H3 recruiting HPI and Polycomb, respectively) directly precede the phospho-acceptor. In contrast, the phospho-acceptor comes first in the activating Thr 3/Lys 4 residue pair in the H3 tail. From these observations, a “order-first” rule is hypothesized wherein “lysine-first” “methyl/phos switches” (LysSer/Thr) dictate a silenced or “off” state of chromatin. In contrast, “serine/threonine-first” “phos/methyl switches” (Ser/ThrLys) may regulate activated or “on” states of chromatin (See FIG. 3). The Thr 22/Lys 23 pair in H3 might therefore represent a previously unrecognized activating “methyl/phos switch.” The situation is, however, less clear at the “silencing clusters” in H3 (Lys 79/Thr 80) and H4 (Lys 79/Thr 80), and methylation at Lys 79 might represent an exception to the proposed “order-first” rule. If it is indeed an “on” mark reducing SIR repressor binding, loss of silencing in yeast mutants of H3 Lys 79 and the corresponding HMT, Dot1, must be caused by indirect effects.

The proposed “order-first” rule might only apply to more-stable marks such as lysine methylation. Acetylation of Lys 9 in H3, for example, is well documented. Several studies have shown that acetyl-Lys 9 synergizes with phospho-Ser 10, and the di-modified, acetyl/phos state of this pair correlates with immediate-early gene activation. However, a phospho-switch (i.e. a “release button” for methyl-binding effector proteins) may not be needed to “eject” acetyl-binding modules (such as bromodomains), because acetyl-marks are easily erased by HDAC activities. Similar considerations might apply to sites of ubiquitination, as deubiquitinating enzymes exist. Yet Lys 119 of H2A precedes Thr 120, which would be consistent with this ubiquityl mark functioning as an “off” switch. Indeed, recent work shows that ubiquitination of H2A Lys 119 is enriched in the heterochromatic XY sex body during murine spermatogenesis. In contrast, Lys 120 of H2B is preceded by Thr 119, which would be consistent with this ubiquityl mark functioning as an “on” switch. In support, ubiquitination of H2B governs an activating “trans-histone” regulatory pathway in yeast that involves methylation of Lys 4 and Lys 79 in H3. Moreover, ubiquitinated H2B is only detected in transcriptionally active macronuclei of Tetrahymena and is absent from transcriptionally silent micronuclei that contain ubiquitinated H2A.

The proposed “order-first” rule of “lysine-first” (off) versus “serine/threonine-first” (on) switches (See FIG. 3) could be implemented based on the fact that the binding of most modules to their cognate marks is highly asymmetric.

For example, the chromodomains of HP1 and Polycomb make contacts to four to six residues N-terminal to the recognized methyl-lysine marks, but “see” only one residue C-terminal to the modification site. Similarly, the structure of the bromodomain of Gcn5 bound to the H4 Lys 16 acetyl-mark shows major contacts only to the N-terminal face of the binding site. Indeed, asymmetry of binding interfaces is a common phenomenon in cell biology (for example, SH2 domains bind asymmetrically to sequences containing phospho-tyrosine). An asymmetric readout of switch-sites by conserved modules could dictate the biology of these post-translational modifications. Modules that read the “on” switches (like Thr 3 Lys 4^{methyl}) or the “off” switches (like Lys 9^{methyl} Ser 10, Lys 27^{methyl} Ser 28) of the “order-first” rule might therefore recognize opposed and contrary faces of their cognate binding sites.

Multiple Dimensions/Layers of the “Histone Code”

The “histone code” is a multi-dimensional problem, representing several layers of regulation including modification patterns on single histones (first layer/dimension, “marks” and “modification cassettes”), interrelationships within a single nucleosome core (second layer/dimension, “modification matrix”), and on defined nucleosomal or chromosomal domains (third level/dimension). Interrelationships between nucleosomal or chromosomal domains may be mediated and read by different mechanisms. Effector modules and histone modifying complexes could be recruited by certain marks but excluded/repelled by other modifications. Effectors or effector complexes that contain more than one recognition module for a certain modification (or modification pattern) could mediate long-range effects. Such binding factors could serve as “bridging clamps” to bring together and potentially anchor distant nucleosomal arrays. In addition, modifying enzymes that contain binding modules or bind to effectors could reinforce and expand the modification pattern to adjacent nucleosomes (chromatin/histone modifiers). Because of the dynamic nature of histone modifications, time has to be considered as an additional (fourth dimension/layer) of the “histone code.” Depending on the physiological internal and external stimuli that manifest in different “modification matrices”, each combination of “marks” could theoretically define a particular state of chromatin at a given stage in the life cycle of a cell or organism.

Histone Modification Matrix

The platform of the “histone code” is unlikely to be single histones or single histone tails, but rather the “modification cassette” concept must be expanded to better reflect the multiple dimensions of chromatin itself (nucleosomes and arrays of nucleosomes). The concept of a “modification matrix” is used to describe the complexity of the “histone code” on the level of a single nucleosome. The value of this concept can best be illustrated in the context of silencing clusters (three-dimensional patches of H3 and H4) recently identified by genetic screens in yeast, the sin mutations, and the collateral effect of histone ubiquitination on multiple and selective histone methylation events.

Furthermore, the combination of dense marks in short clusters, situated at strategic locations in the histones, could form defined “modification cassettes” with distinct biological readouts depending on their modification state. Short “cassettes” where at least three out of five sites separated by no more than one residue can be covalently modified are readily spotted in the H3 and H4 tails. The stretch between Lys 9 and Thr 11 of H3, for example, can carry four different covalent marks. Additional clusters of adjacent marks on H3 are centred around Lys 4 and Lys 27. On the H4 tail, “cassettes” where single modification sites are separated by only one residue can be found. In H4 of most species, Ser 1, Arg 3, and Lys 5 are well-known sites of phosphorylation, methylation and acetylation, respectively. Similarly, Lys 16 and Lys 20, situated more internally in the H4 tail, are sites of acetylation and methylation, respectively, and an older literature suggests that His 18 might be an acid-labile phosphorylation site. It is anticipated that the modification of adjacent sites within short clusters of accumulated histone modifications will influence the recognition and binding of modules to their cognate mark. Especially since the recognition of short clusters of 5-10 amino acids is a hallmark of the interaction of most modules with their target interaction marks. Furthermore, modules that directly read complex patterns of marks might exist.

Implications beyond Histones

Some putative “modification cassettes” of major histones are also found in minor histone variants, possibly arguing for conserved functions. For example, the extreme N terminus of H4 in most species is also found at the very N terminus of most, but not all, H2A variants. In contrast, the stretch [AG-GK . . . ], which marks the beginning of the H2A.Z variant in several species, is identical to the extreme N terminus of Tetrahymena H4. Could the change of Ser1 to Ala1 be linked to the loss of Arg3 in specific members of both H2A and H4 gene families? Perhaps phosphorylation at Ser1 in H4 or H2A is linked to methylation at nearby Arg3 (or vice versa).

The development of sensitive proteomic approaches is likely to lead to the discovery of additional clusters of modifications on other non-histone proteins. Comparison of the sequence motifs of the proposed “modification cassettes” with other proteins identifies some striking similarities. For example, the extreme N terminus of most H4 proteins [SGRGK . . . ] is found precisely in the AML/RUNX/RUNT family of transcriptional regulators, and this motif is conserved in sequences from fly to human. Similarly, the Drosophila H4 N-terminal motif [TGRGK . . . ] is also present in the NRF and MCM4 proteins of higher organisms and at the very N terminus of Drosophila Polycomb. While it remains unknown to what extent covalent modifications exist in these short clusters, it is anticipated that these conserved motifs could represent discrete information units with yet unknown regulatory function. Whether the general concepts presented here for histones might ultimately be applicable to other proteins and signalling cascades remains an intriguing possibility.

Redundancy of Histone Tails and Histone-Tail Marks

It is well documented that the histone H3 tail is functionally redundant with that of H4 in yeast. Similarly, the tail of H2A is redundant with that of H2B. The general inability to generate clear phenotypes from mutations in highly conserved, and in some cases, invariant sites of post-translational modification suggests that functional redundancy exists between histone covalent marks on different tails. For example, mutations at mitotic phosphorylation sites in H3 (S10A, S28A or S10,28A) fail to yield mitotic phenotypes in budding yeast. In contrast, the S10A mutation in Tetrahymena H3 exhibited clear defects in chromosome condensation and segregation. The recent finding that Ser1 in the H4 tail is a strong mitotic/meiotic phosphorylation site in organisms ranging from yeast to man, combined with the fact that Ser1 is naturally an alanine in Tetrahymena H4 supports the notion that functional redundancy exists between histone covalent marks on different tails. However, it remains unclear if such redundancy is a common event or not.

Also informative is the situation of Lys4 methylation within the H3 tail. In yeast, mutation of this site (K4R), or disruption of the gene encoding the Lys4 methyltransferase Set1, yield a modest slow-growth phenotype on rich media. Interestingly, mutation of the same site in Tetrahymena H3 (K4R) is lethal. Most H4s contain Arg3, a site of methylation that, like Lys4 methylation is generally recognized to be an “on” mark in chromatin. Arg3 is missing in the Tetrahymena H4 tail, and thus, this methylation mark can not occur in this organism. We predict then that H3 (Lys4) methylation is functionally redundant with H4 (Arg3) methylation and that this fact may explain the difference in phenotype between H3 K4R mutations in yeast and Tetrahymena. If H4 (Arg3) methylation compensates for the loss of H3 Lys4 methylation in yeast, this can not occur in Tetrahymena producing an inviable cell via the loss of a critical “on” mark. In support, HSL7 (Histone Synthetic Lethal 7), encoding a putative Arg methyltransferase in yeast, was identified in a genetic screen looking for mutations that are synthetically lethal with a “tailess” H3 strain. It is of interest to know if Hs17 is a yeast H4 Arg3 methyltransferase, if the H3 K4R/H4 R3K double mutant is lethal in yeast, and if H3 K4R mutants (or set1 disruption strains) are synthetically lethal when combined with “tailess” H4 strains.

Less well studied are the post-translational marks on histone H2A and H2B. Nonetheless, the idea that covalent marks on one tail may be functionally redundant with those on another tail dictates the need for experiments where mutations in modification sites can be generated in all possible combinations. Unexpected surprises, including the recent finding for unidirectional, “trans-tail” regulation of one histone mark for another (i.e. ubiquitination of the C-tail of H2B governs the methylation of Lys4 (and Lys79) in the H3 tail), underscores the sequential and concerted nature of the histone code as it is written using a language beyond what is contained within a linear (one-dimensional) histone tail.

Histone Variants

In addition to the versatility and staggering potential combinatorial possibilities of distinct modification “marks” in the major histone types, histone variants (subtypes/isoforms) add another layer of complexity to the “histone code” that has yet to be fully understood or appreciated. Several lines of evidence suggest that histone variants add another meaningful layer to the overall complexity and regulatory options to the histone code. First, the mere existence of histone variants adds further plasticity to the “histone code” by changing the platform upon which the code is written. However, in some organisms what is a minor histone subtype is another organism's principal histone form. For example, consider that H2A.X is a relatively minor histone variant in mammals, while in budding yeast, its major form of H2A most resembles this variant. In as much as phosphorylation of a C-terminal Ser139 in mammalian H2A.X is often associated with DNA damage, it seems likely that yeast imparted this function into its major H2A.

However, in most organisms, little work has been done on the post-translational modifications of histone variants in part because of their often lower abundance in bulk chromatin. Still, using evolution as a guide, it seems likely, if not certain, that histone variants will have unique patterns of covalent modifications. For example, H3.3 has a unique serine at position 31 (an alanine in general H3) suggesting that phosphorylation at this position may be one distinguishing feature of this variant from the major form of H3. Ser14 recently identified to be an apoptotic phosphorylation mark in vertebrate H2Bs has been converted to a phenylalanine in a testis-specific form of H2B that is conserved between mouse and humans. In as much phosphorylation of H2B at Ser14 is strongly correlated with apoptotic chromatin condensation and/or aggregation, it seems likely that this phosphorylation function has been lost in the testis H2B variants.

Finally, it is note that an “acidic patch” found in many H2As is interrupted by a phosphorylation site in many of the H2A.Z family members. Interestingly, this acidic patch has been suggested to interact with a “basic patch” in the H4 tail [KRHRK] that is a potential histone modification cassette. If this acidic patch in H2A does interact in higher-order chromatin with the basic patch on the H4 tail, these potential phosphorylation and/or acetylation sites may be used to “unzip” these “polar zippers.” One testable function for the H2A.Z histone variants is that they may have evolved a distinct surface interaction with H4 that can be “unglued” from H4 by reversible phosphorylation at this additional phosphorylation site. Variants are deposited to specific regions or locations of chromatin and thus these varients provide a means to change the histone code in specific genomic locations. For example, the H3 variant CENP-A is found only at centromeres providing a mechanism to selectively recruit distinct machinery to this chromosomal region.

Histone variants can be selectively incorporated into non-replicating chromatin (e.g. the deposition of H3.3, which seems to coincide with transcriptionally active regions). Non-replication dependent exchange of histones from chromatin could establish an alternative route of erasing or exchanging modification patterns to reprogram chromatin networks. Active exchange of histone variants may provide at least a partial solution to the problem of how unwanted methyl marks are removed from chromatin if active histone demethylases are rare or non-existent. It can be argued that evolution has introduced very little change in the histone proteins, because of their fundamental role in DNA packaging. However, it is well accepted that histone tails are generally dispensable for nucleosome folding, and yet these domains are also highly conserved. In some cases, sites of reversible modification, like the well-known acetylation sites in H4, are invariant.

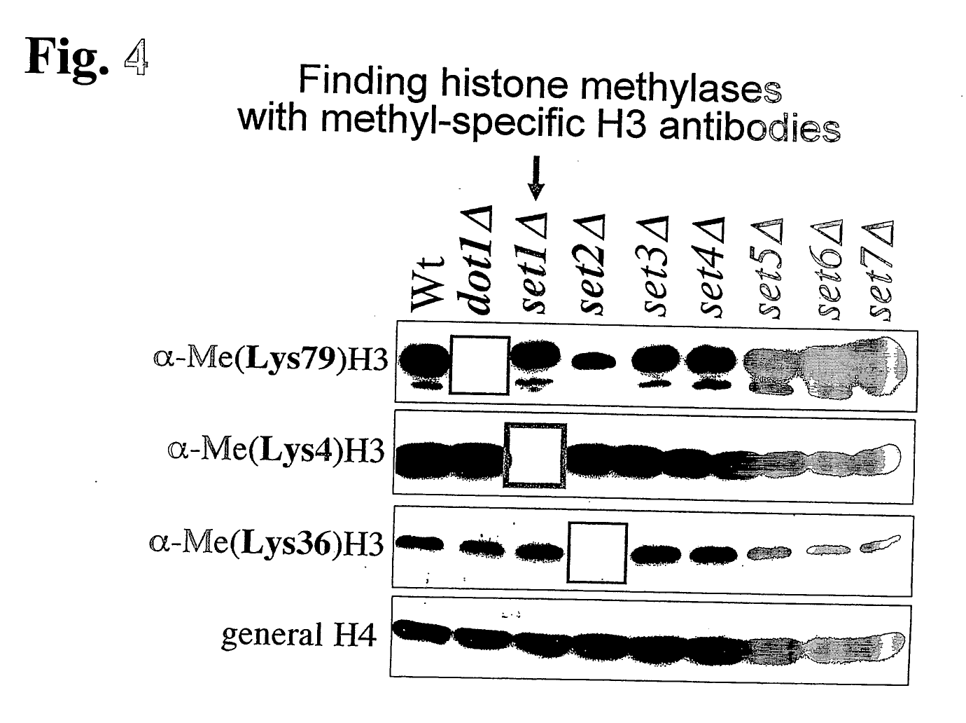

In accordance with one embodiment of the present invention antibodies to specific histone modifications can be used to identify the function of non-essential enzymes. For example, as shown in FIG. 4, the methylating activity of several disrupted genes (dot1, set2 and set 1) can be identified based on an antibody probing of whole cell yeast extracts, provided that the target gene, when disrupted, produces a viable yeast cell. Loss of the function of the disrupted target gene results in the loss of the posttranslational modification on the substrate protein (e.g. a histone protein) and the absence of the modification is detected by the failure of the relevant modification specific antibody to bind to a Western blot of mutant cells' proteins. Note the results shown in FIG. 4 identify the yeast genes responsible for methylating the following lysine residues in H3: Lys79 (dot1), Lys36 (set2) and Lys4 (set1). The closest human homolog of Set1 in yeast is human MLL1. The SET domain of MLL1 is involved with Lys4 methylation and gene regulation in mouse HOX genes. Importantly, MLL stands for Mixed Lineage Leukemia gene, this gene is involved in may acute forms of human leukemias when its function is dysfunctional.

The function of yeast genes Set3-Set7 has yet to be elucidated, but they are likely to be methyltransferases. Perhaps they will be histone methyltransferases (HMTs) as well. In accordance with one embodiment of the present invention histones from all of these viable yeast strains are isolated and probed with the appropriate antibody (one specific for the methlated histones) to determine what methyl mark is missing. In wildtype cells, one would anticipate that most methyl marks will be present. In set1 knock-out strains, the Lys 4 methyl of H3 will be missing. Thus, there are elegant positive and negative controls to double check the technology. Furthermore, subtractive comparisons can be conducted between two samples to tell what is in common and what is different. More particularly, one aspect of the present invention is directed to identifying the substrates of known non-lethal yeast mutations by looking to see what histone post-tranlational modification is missing in the mutant yeast cell.

In accordance with one embodiment a method of identifying the function/substrate of a non-essential posttranslational amino acid modifying enzyme is provided. The method comprises the steps of isolating peptides from an organism that is defective in the expression of a non-essential posttranslational amino acid modifying enzyme. The isolated peptides are then separated by electrophoresis, and transferred a membrane and then probed with an antibody that is specific for a known posttranslational mark present in wild type strains of the organism. If the antibody fails to bind to the proteins isolated from the mutant strain, but binds to proteins isolated from the wild type strain, then the modification the antibody typically binds to is likely the natural substrate of the native enzyme that correspondes to the enzyme disrupted in the mutant strain.

In accordance with one embodiment of the present invention a method of detecting modified histone proteins is provided. The method comprises the steps of contacting histone proteins with an antibody, wherein the antibody specifically binds only to histones that comprise a modified sequence selected from the group consisting of:

| | |
|---|---|
| ART^{(P)}KQ, | (SEQ ID NO: 1) |
| ART^{(P)}K^{(M)}Q, | (SEQ ID NO: 2) |
| ARKS^{(P)}T, | (SEQ ID NO: 3) |
| ARK^{(M)}S^{(P)}T, | (SEQ ID NO: 4) |
| LAT^{(P)}KA, | (SEQ ID NO: 5) |
| LAT^{(P)}K^{(M)}A, | (SEQ ID NO: 6) |
| LATK^{(M)}A, | (SEQ ID NO: 7) |
| ARKS^{(P)}A, | (SEQ ID NO: 8) |
| ARK^{(M)}S^{(P)}A, | (SEQ ID NO: 9) |
| ARK^{(M)}SA, | (SEQ ID NO: 10) |
| PKKT^{(P)}E, | (SEQ ID NO: 11) |
| PKK^{(U)}T^{(P)}E, | (SEQ ID NO: 12) |
| PKK^{(U)}TE, | (SEQ ID NO: 13) |
| AVT^{(P)}KY, | (SEQ ID NO: 14) |
| AVT^{(P)}K^{(U)}Y, | (SEQ ID NO: 15) |
| AVTK^{(U)}Y, | (SEQ ID NO: 16) |
| DFK^{(M)}TD, | (SEQ ID NO: 20) |
| DFK^{(M)}T^{(P)}D, | (SEQ ID NO: 21) |
| DFKT^{(P)}D, | (SEQ ID NO: 22) |
| KRK^{(M)}TV, | (SEQ ID NO: 23) |
| KRK^{(M)}T^{(P)}V, | (SEQ ID NO: 24) |
| and | |
| KRKT^{(P)}V. | (SEQ ID NO: 25) |

The antibodies of the present invention can be linked to a detectable label using standard reagents and techniques known to those skilled in the art. For example, see Wensel and Meares, Radioinmunoimaging and Radioimmunotherapy, Elsevier, New York (1983), which is hereby incorporated by reference, for techniques relating to the radiolabeling of antibodies. See also, D. Colcher et al., “Use of Monoclonal Antibodies as Radiopharmaceuticals for the Localization of Human Carcinoma Xenografts in Athymic Mice,” Meth. EnzvmQL, 121: 802-816 (1986), which is hereby incorporated by reference.

Because the antibodies of the present invention have the potential for use in humans as diagnostic and therapeutic agents, one embodiment of the present invention is directed to humanized versions of these antibodies. Humanized versions of the antibodies are needed for therapeutic applications because antibodies from non-human species may be recognized as foreign substances by the human immune system and neutralized such that they are less useful. Humanized antibodies are immunoglobulin molecules comprising a human and non-human portion. More specifically, the antigen combining region (variable region) of a humanized antibody is derived from a non-human source (e.g. murine) and the constant region of the humanized antibody is derived from a human source. The humanized antibody should have the antigen binding specificity of the non-human antibody molecule and the effector function conferred by the human antibody molecule. Typically, creation of a humanized antibody involves the use of recombinant DNA techniques.

In accordance with one embodiment, the antibodies of the present invention are attached to a solid support and used to immunoprecipitate chromatin. In one embodiment the antibodies that specifically bind to a polypeptide comprising an amino acid sequence selected from the group consisting of SEQ ID NOs 1-16, and 20-25 are linked to a synthetic solid support. In another embodiment one or more polypeptides, comprising an amino acid sequence selected from the group consisting of SEQ ID NOs 1-16, and 20-25 are attached to a solid support and contacted with cell extracts to capture natural ligands (i.e. effectors) of the modified peptides. The support may be in particulate or solid form and in one embodiment is a synthetic support including, but is not limited to: a plate, a test tube, beads, a ball, a filter or a membrane. Methods for fixing antibodies/proteins to insoluble synthetic supports are known to those skilled in the art.

In one embodiment an antibody of the current invention is fixed to an insoluble support that is suitable for use in affinity chromatography.

Immunoprecipitation of chromatin will be used in one embodiment of the invention to map the location of DNA-binding proteins at a genome-wide level through the use of microarrays. In addition, chromatin immunoprecipitation assays, using modification-specific histone antibodies, can be used to analyze a wide range of DNA-templated processes that are governed by the chromatin environment.

In one embodiment of the present invention a kit is provided for determining the status of a binary switch (i.e. is the site in an effector binding state or not). The kit comprises an antibody that specifically binds to an amino acid sequence selected from the group consisting of:

| | |
|---|---|
| ART^{(P)}KQ, | (SEQ ID NO: 1) |
| ART^{(P)}K^{(M)}Q, | (SEQ ID NO: 2) |
| ARKS^{(P)}T, | (SEQ ID NO: 3) |
| ARK^{(M)}S^{(P)}T, | (SEQ ID NO: 4) |
| LAT^{(P)}KA, | (SEQ ID NO: 5) |
| LAT^{(P)}K^{(M)}A, | (SEQ ID NO: 6) |
| LATK^{(M)}A, | (SEQ ID NO: 7) |
| ARKS^{(P)}A, | (SEQ ID NO: 8) |
| ARK^{(M)}S^{(P)}A, | (SEQ ID NO: 9) |
| ARK^{(M)}SA, | (SEQ ID NO: 10) |
| ARTK^{(M)}Q, | (SEQ ID NO: 18) |
| ARK^{(M)}ST, | (SEQ ID NO: 19) |
| DFK^{(M)}TD, | (SEQ ID NO: 20) |
| DFK^{(M)}T^{(P)}D, | (SEQ ID NO: 21) |
| DFKT^{(P)}D, | (SEQ ID NO: 22) |
| KRK^{(M)}TV, | (SEQ ID NO: 23) |
| KRK^{(M)}T^{(P)}V, | (SEQ ID NO: 24) |
| and | |
| KRKT^{(P)}V. | (SEQ ID NO: 25) |

In one embodiment the kit comprises a first antibody that specifically binds to ART^{(P)}K^{(M)}Q (SEQ ID NO: 2), and a second antibody that specifically binds to ARTK^{(M)}Q (SEQ ID NO: 18). In another embodiment the kit comprises a first antibody that specifically binds to ARK^{(M)}S^{(P)}T (SEQ ID NO: 4), and a second antibody that specifically binds to ARK^{(M)}ST (SEQ ID NO: 19). In another embodiment the kit comprises a first antibody that specifically binds to LAT^{(P)}K^{(M)}A (SEQ ID NO: 6), and a second antibody that specifically binds to LATK^{(M)}A (SEQ ID NO: 7). In another embodiment the kit comprises a first antibody that specifically binds to ARK^{(M)}S^{(P)}A (SEQ ID NO: 9), and a second antibody that specifically binds to ARK^{(M)}SA (SEQ ID NO: 10).

In one embodiment the antibodies are attached to a synthetic solid support, wherein the support is either a monolithic solid or is in particular form. In one preferred embodiment the antibodies are monoclonal antibodies and in a further embodiment the antibodies are labeled. To this end, the antibodies of the present invention can be packaged in a variety of containers, e.g. vials, tubes, microtiter well plates, bottles, and the like. Other reagents can be included in separate containers and provided with the kit; e.g., positive control samples, negative control samples, buffers, cell culture media, etc.

The kits of the present invention may further comprise reagents for detecting the monoclonal antibody once it is bound to the target antigen. Optionally, reagents (pepsin, dilute hydrochloric acid) for treating cells or tissue to render nuclear proteins accessible for immunological binding may also be included, as may immunofluorescent detection reagents (an anti-immunoglobulin antibody derivatized with fluorescein or rhodamine, or a biotinylated anti-immunoglobulin antibody together with avidin or streptavidin derivatized with fluorescein or rhodamine), immunohistochemical or immunocytochemical detection reagents (an anti-immunoglobulin antibody derivatized with alkaline phosphatase or horseradish peroxidase, or a biotinylated anti-immunoglobulin antibody together with avidin or streptavidin derivatized with alkaline phosphatase or horseradish peroxidase). In one embodiment, the kit includes one or more reagents for immunoperoxidase staining (an anti-immunoglobulin antibody derivatized with horseradish peroxidase, or a biotinylated anti-immunoglobulin antibody together with avidin or streptavidin derivatized with horseradish peroxidase), together with a chromogenic substrate therefor (e.g., diaminobenzidine).

1. A purified antibody that specifically binds to a polypeptide comprising an amino acid sequence selected from the group consisting of:

| | |
|---|---|
| ART^{(P)}KQ, | (SEQ ID NO: 1) |
| ART^{(P)}K^{(M)}Q, | (SEQ ID NO: 2) |
| ARKS^{(P)}T, | (SEQ ID NO: 3) |
| ARK^{(M)}S^{(P)}T, | (SEQ ID NO: 4) |
| LAT^{(P)}KA, | (SEQ ID NO: 5) |
| LAT^{(P)}K^{(M)}A, | (SEQ ID NO: 6) |
| LATK^{(M)}A, | (SEQ ID NO: 7) |
| ARKS^{(P)}A, | (SEQ ID NO: 8) |
| ARK^{(M)}S^{(P)}A, | (SEQ ID NO: 9) |
| ARK^{(M)}SA, | (SEQ ID NO: 10) |
| PKKT^{(P)}E, | (SEQ ID NO: 11) |
| PKK^{(U)}T^{(P)}E, | (SEQ ID NO: 12) |
| PKK^{(U)}TE, | (SEQ ID NO: 13) |
| AVT^{(P)}KY, | (SEQ ID NO: 14) |
| AVT^{(P)}K^{(U)}Y, | (SEQ ID NO: 15) |
| and | |
| AVTK^{(U)}Y. | (SEQ ID NO: 16) |

2. The antibody of claim 1 wherein the antibody specifically binds to a polypeptide comprising an amino acid sequence selected from the group consisting of

| | |
|---|---|
| ART^{(P)}KQ, | (SEQ ID NO: 1) |
| ART^{(P)}K^{(M)}Q, | (SEQ ID NO: 2) |
| ARKS^{(P)}T, | (SEQ ID NO: 3) |
| ARK^{(M)}S^{(P)}T, | (SEQ ID NO: 4) |
| LAT^{(P)}KA, | (SEQ ID NO: 5) |
| LAT^{(P)}K^{(M)}A, | (SEQ ID NO: 6) |
| LATK^{(M)}A, | (SEQ ID NO: 7) |
| ARKS^{(P)}A, | (SEQ ID NO: 8) |
| ARK^{(M)}S^{(P)}A, | (SEQ ID NO: 9) |
| and | |
| ARK^{(M)}SA. | (SEQ ID NO: 10) |

3. The antibody of claim 1 wherein the antibody specifically binds to a polypeptide selected from the group consisting of

| | |
|---|---|
| PKKT^{(P)}E | (SEQ ID NO: 11) |
| PKK^{(U)}T^{(P)}E, | (SEQ ID NO: 12) |
| PKK^{(U)}TE, | (SEQ ID NO: 13) |
| AVT^{(P)}KY, | (SEQ ID NO: 14) |
| AVT^{(P)}K^{(U)}Y, | (SEQ ID NO: 15) |
| and | |
| AVTK^{(U)}Y. | (SEQ ID NO: 16) |

4. The antibody of claim 1 wherein the antibody is a monoclonal antibody.

5. The antibody of claim 4, wherein the antibody specifically binds to a polypeptide comprising the sequence PKK^{(U)}TE (SEQ ID NO: 13) or AVTK^{(U)}Y (SEQ ID NO: 16).

6. A composition comprising the antibody of claim 1 and a diluent or pharmaceutically acceptable carrier.

7. The antibody of claim 4 wherein said antibody is coupled to a protein.

8. The antibody of claim 4 wherein said antibody is coupled to a chemotherapeutic agent.

9. The antibody of claim 4, wherein the antibody is labeled.

10. The antibody of claim 9, wherein the antibody is labeled with a fluorescent marker.

11. The antibody of claim 4 wherein said antibody is linked to a solid support.

12. An antigenic peptide consisting of 5 to 20 amino acids, said peptide comprising an amino acid sequence selected from the group consisting of:

| | |
|---|---|
| ART^{(P)}KQ, | (SEQ ID NO: 1) |
| ART^{(P)}K^{(M)}Q, | (SEQ ID NO: 2) |
| ARKS^{(P)}T, | (SEQ ID NO: 3) |
| ARK^{(M)}S^{(P)}T, | (SEQ ID NO: 4) |
| LAT^{(P)}KA, | (SEQ ID NO: 5) |
| LAT^{(P)}K^{(M)}A, | (SEQ ID NO: 6) |
| LATK^{(M)}A, | (SEQ ID NO: 7) |
| ARKS^{(P)}A, | (SEQ ID NO: 8) |
| ARK^{(M)}S^{(P)}A, | (SEQ ID NO: 9) |
| ARK^{(M)}SA, | (SEQ ID NO: 10) |
| PKKT^{(P)}E, | (SEQ ID NO: 11) |
| PKK^{(U)}T^{(P)}E, | (SEQ ID NO: 12) |
| PKK^{(U)}TE, | (SEQ ID NO: 13) |
| AVT^{(P)}KY, | (SEQ ID NO: 14) |
| AVT^{(P)}K^{(U)}Y, | (SEQ ID NO: 15) |
| and | |
| AVTK^{(U)}Y. | (SEQ ID NO: 16) |

13. The antigenic peptide of claim 12 wherein said peptide comprises an amino acid sequence selected from the group consisting of

| | |
|---|---|
| ART^{(P)}KQ, | (SEQ ID NO: 1) |
| ART^{(P)}K^{(M)}Q, | (SEQ ID NO: 2) |
| ARKS^{(P)}T, | (SEQ ID NO: 3) |
| ARK^{(M)}S^{(P)}T, | (SEQ ID NO: 4) |
| LAT^{(P)}KA, | (SEQ ID NO: 5) |
| LAT^{(P)}K^{(M)}A, | (SEQ ID NO: 6) |
| LATK^{(M)}A, | (SEQ ID NO: 7) |
| ARKS^{(P)}A, | (SEQ ID NO: 8) |
| ARK^{(M)}S^{(P)}A, | (SEQ ID NO: 9) |
| and | |
| ARK^{(M)}SA. | (SEQ ID NO: 10) |

14. The antigenic peptide of claim 12 wherein said peptide comprises an amino acid sequence selected from the group consisting of

| | |
|---|---|
| PKKT^{(P)}E, | (SEQ ID NO: 11) |
| PKK^{(U)}T^{(P)}E, | (SEQ ID NO: 12) |
| PKK^{(U)}TE, | (SEQ ID NO: 13) |
| AVT^{(P)}KY, | (SEQ ID NO: 14) |
| AVT^{(P)}K^{(U)}Y, | (SEQ ID NO: 15) |
| and | |
| AVTK^{(U)}Y. | (SEQ ID NO: 16) |

15. A composition comprising the peptide of claim 12 and a pharmaceutically acceptable carrier.

16. The composition of claim 15 further comprising an adjuvant.

17. A diagnostic test kit for detecting euchromatin and heterochromatin, said kit comprising an antibody that specifically binds to a polypeptide comprising an amino acid sequence selected from the group consisting of

| | |
|---|---|
| ART^{(P)}KQ, | (SEQ ID NO: 1) |
| ART^{(P)}K^{(M)}Q, | (SEQ ID NO: 2) |
| ARKS^{(P)}T, | (SEQ ID NO: 3) |
| ARK^{(M)}S^{(P)}T, | (SEQ ID NO: 4) |
| LAT^{(P)}KA, | (SEQ ID NO: 5) |
| LAT^{(P)}K^{(M)}A, | (SEQ ID NO: 6) |
| LATK^{(M)}A, | (SEQ ID NO: 7) |
| ARKS^{(P)}A, | (SEQ ID NO: 8) |
| ARK^{(M)}S^{(P)}A, | (SEQ ID NO: 9) |
| and | |
| ARK^{(M)}SA. | (SEQ ID NO: 10) |

18. The kit of claim 17 wherein said kit comprises a first antibody that specifically binds to ART^{(P)}K^{(M)}Q (SEQ ID NO: 2), and a second antibody that specifically binds to ARTK^{(M)}Q (SEQ ID NO: 18).

19. The kit of claim 17 wherein said kit comprises a first antibody that specifically binds to ARK^{(M)}S^{(P)}T (SEQ ID NO: 4), and a second antibody that specifically binds to ARK^{(M)}ST (SEQ ID NO: 19).

20. The kit of claim 17 wherein said kit comprises a first antibody that specifically binds to LAT^{(P)}K^{(M)}A (SEQ ID NO: 6), and a second antibody that specifically binds to LATK^{(M)}A (SEQ ID NO: 7).

21. The kit of claim 17 wherein said kit comprises a first antibody that specifically binds to ARK^{(M)}S^{(P)}A (SEQ ID NO: 9), and a second antibody that specifically binds to ARK^{(M)}SA (SEQ ID NO: 10).

22. A method of identifying a postranslational amino acid modifying enzyme, said method comprising the steps of
isolating peptides from an organism that is defective in the expression of an enzymatic protein;
separating the isolated peptides by gel electrophoresis;
probing the separated peptides with an antibody specific for a known posttranslational mark present in the wild type strain of the organism; wherein the absence of binding of the antibody to the separated peptides identifies said enzymatic protein as having the function of creating said posttranslational mark in wild type organisms.

23. The method of claim 22 wherein the posttranslational mark is a methylated amino acid.

24. A method of detecting chromatin alterations that are associated with a disease state, said method comprising the steps of
isolating chromatin from both normal and diseased tissue to create a first and second pool of chromatin;
contacting the first and second pools of chromatin with an antibody that specifically binds to a polypeptide selected from the group consisting of

| | |
|---|---|
| ART^{(P)}KQ, | (SEQ ID NO: 1) |
| ART^{(P)}K^{(M)}Q, | (SEQ ID NO: 2) |
| ARKS^{(P)}T, | (SEQ ID NO: 3) |
| ARK^{(M)}S^{(P)}T, | (SEQ ID NO: 4) |
| LAT^{(P)}KA, | (SEQ ID NO: 5) |
| LAT^{(P)}K^{(M)}A, | (SEQ ID NO: 6) |
| LATK^{(M)}A, | (SEQ ID NO: 7) |
| ARKS^{(P)}A, | (SEQ ID NO: 8) |
| ARK^{(M)}S^{(P)}A, | (SEQ ID NO: 9) |
| ARK^{(M)}SA, | (SEQ ID NO: 10) |
| PKKT^{(P)}E, | (SEQ ID NO: 11) |
| PKK^{(U)}T^{(P)}E, | (SEQ ID NO: 12) |
| PKK^{(U)}TE, | (SEQ ID NO: 13) |
| AVT^{(P)}KY, | (SEQ ID NO: 14) |
| AVT^{(P)}K^{(U)}Y, | (SEQ ID NO: 15) |
| and | |
| AVTK^{(U)}Y; | (SEQ ID NO: 16) |
| and | |

comparing the staining pattern of the antibody bound chromatin isolated from normal tissue to the staining pattern of the antibody bound chromatin isolated from the diseased tissue.

25. The method of claim 24 further comprising the step of fragmenting the isolated chromatin before the immunoprecipitation step.

26. A purified antibody that specifically binds to a polypeptide comprising an amino acid sequence selected from the group consisting of:

| | |
|---|---|
| DFK^{(M)}TD, | (SEQ ID NO: 20) |
| DFK^{(M)}T^{(P)}D, | (SEQ ID NO: 21) |
| DFKT^{(P)}D, | (SEQ ID NO: 22) |
| KRK^{(M)}TV, | (SEQ ID NO: 23) |
| KRK^{(M)}T^{(P)}V, | (SEQ ID NO: 24) |
| and | |
| KRKT^{(P)}V. | (SEQ ID NO: 25) |

27. An antigenic peptide consisting of 5 to 20 amino acids, said peptide comprising an amino acid sequence selected from the group consisting of:

| | |
|---|---|
| DFK^{(M)}TD, | (SEQ ID NO: 20) |
| DFK^{(M)}T^{(P)}D, | (SEQ ID NO: 21) |
| DFKT^{(P)}D, | (SEQ ID NO: 22) |
| KRK^{(M)}TV, | (SEQ ID NO: 23) |
| KRK^{(M)}T^{(P)}V, | (SEQ ID NO: 24) |
| and | |
| KRKT^{(P)}V. | (SEQ ID NO: 25) |

28. A diagnostic test kit for detecting euchromatin and heterochromatin, said kit comprising an antibody that specifically binds to a polypeptide comprising an amino acid sequence selected from the group consisting of

| | |
|---|---|
| DFK^{(M)}TD, | (SEQ ID NO: 20) |
| DFK^{(M)}T^{(P)}D, | (SEQ ID NO: 21) |
| DFKT^{(P)}D, | (SEQ ID NO: 22) |
| KRK^{(M)}TV, | (SEQ ID NO: 23) |
| KRK^{(M)}T^{(P)}V, | (SEQ ID NO: 24) |
| and | |
| KRKT^{(P)}V. | (SEQ ID NO: 25) |

29. The kit of claim 27 comprising a first antibody that specifically binds to DFK^{(M)}T^{(P)}D (SEQ ID NO: 21), and a second antibody that specifically binds to DFK^{(M)}TD (SEQ ID NO: 20).

30. The kit of claim 28 comprising a first antibody that specifically binds to KRK^{(M)}T^{(P)}V (SEQ ID NO: 24), and a second antibody that specifically binds to KRK^{(M)}TV (SEQ ID NO: 23).


SEQUENCE LISTING (25 sequences)

SEQ ID NO: 1 - protein, 5 aa, Homo sapiens
  MOD_RES (3)..(3): PHOSPHORYLATION
ARTKQ

SEQ ID NO: 2 - protein, 5 aa, Homo sapiens
  MOD_RES (3)..(3): PHOSPHORYLATION
ARTKQ

SEQ ID NO: 3 - protein, 5 aa, Homo sapiens
  MOD_RES (4)..(4): PHOSPHORYLATION
ARKST

SEQ ID NO: 4 - protein, 5 aa, Homo sapiens
  MOD_RES (3)..(3): METHYLATION
ARKST

SEQ ID NO: 5 - protein, 5 aa, Homo sapiens
  MOD_RES (3)..(3): PHOSPHORYLATION
LATKA

SEQ ID NO: 6 - protein, 5 aa, Homo sapiens
  MOD_RES (3)..(3): PHOSPHORYLATION
LATKA

SEQ ID NO: 7 - protein, 5 aa, Homo sapiens
  MOD_RES (4)..(4): METHYLATION
LATKA

SEQ ID NO: 8 - protein, 5 aa, Homo sapiens
  MOD_RES (4)..(4): PHOSPHORYLATION
ARKSA

SEQ ID NO: 9 - protein, 5 aa, Homo sapiens
  MOD_RES (4)..(4): PHOSPHORYLATION
ARKSA

SEQ ID NO: 10 - protein, 5 aa, Homo sapiens
  MOD_RES (3)..(3): METHYLATION
ARKSA

SEQ ID NO: 11 - protein, 5 aa, Homo sapiens
  MOD_RES (4)..(4): PHOSPHORYLATION
PKKTE

SEQ ID NO: 12 - protein, 5 aa, Homo sapiens
  MOD_RES (3)..(3): Ubiquitination
PKKTE

SEQ ID NO: 13 - protein, 5 aa, Homo sapiens
  MOD_RES (3)..(3): Ubiquitination
PKKTE

SEQ ID NO: 14 - protein, 5 aa, Homo sapiens
  MOD_RES (3)..(3): PHOSPHORYLATION
AVTKY

SEQ ID NO: 15 - protein, 5 aa, Homo sapiens
  MOD_RES (4)..(4): Ubiquitination
AVTKY

SEQ ID NO: 16 - protein, 5 aa, Homo sapiens
  MOD_RES (4)..(4): Ubiquitination
AVTKY

SEQ ID NO: 17 - protein, 5 aa, Homo sapiens
ARTKQ

SEQ ID NO: 18 - protein, 5 aa, Homo sapiens
  MOD_RES (4)..(4): METHYLATION
ARTKQ

SEQ ID NO: 19 - protein, 5 aa, Homo sapiens
  MOD_RES (3)..(3): METHYLATION
ARKSA

SEQ ID NO: 20 - protein, 5 aa, Homo sapiens
  MOD_RES (3)..(3): METHYLATION
DFKTD

SEQ ID NO: 21 - protein, 5 aa, Homo sapiens
  MOD_RES (3)..(3): METHYLATION
DFKTD

SEQ ID NO: 22 - protein, 5 aa, Homo sapiens
  MOD_RES (4)..(4): PHOSPHORYLATION
DFKTD

SEQ ID NO: 23 - protein, 5 aa, Homo sapiens
  MOD_RES (3)..(3): METHYLATION
KRKTV

SEQ ID NO: 24 - protein, 5 aa, Homo sapiens
  MOD_RES (3)..(3): METHYLATION
KRKTV

SEQ ID NO: 25 - protein, 5 aa, Homo sapiens
  MOD_RES (4)..(4): PHOSPHORYLATION
KRKTV